(19) United States
(12) Patent Application Publication
(10) Pub. No.: US 2003/0077759 A1
(43) Pub. Date: Apr. 24, 2003

(54) Plasmids for polyketide production

(76) Inventors: Zhihao Hu, Hayward, CA (US); Richard C. Hutchinson, Cross Plains, WI (US)

Correspondence Address: Brenda J. Wallach, Morrison & Foerster LLP, Suite 500, 3811 Valley Centre Drive, San Diego, CA, 92130-2332, US

(21) Appl. No.: 10/126,196

(22) Filed: Apr. 19, 2002

Related U.S. Application Data
(63) Continuation-in-part of application No. 10/029,368, filed on Dec. 21, 2001, now abandoned.
(60) Provisional application No. 60/259,289, filed on Dec. 28, 2000.

(51) Int. Cl. C12P 19/62; C12N 1/21; C12N 15/74

(52) U.S. Cl. 435/76; 435/320.1; 435/252.3

(57) ABSTRACT

Streptomyces plasmids comprising two SCP2-derived replicons, at least one of which is derived from SCP2@, have a higher copy number and exhibit greater stability than SCP2- or SCP2*-derived plasmids having only a single SCP2-derived replicon and can be used to increase gene expression, relative to the latter plasmids, in host cells.

### CROSS-REFERENCE TO RELATED APPLICATIONS

This application is a continuation-in-part of copending U.S. patent application Ser. No. 10/029,368, filed Dec. 21, 2001, which claims priority to U.S. provisional patent application No. 60/259,289, filed Dec. 28, 2000, now lapsed, both incorporated herein by reference.

### FIELD OF THE INVENTION

The present invention provides recombinant host cells, vectors, and methods for producing useful products, including RNA, proteins, and the products of proteins, such as polyketides and non-ribosomal peptides. Polyketides are a diverse class of compounds with a wide variety of activities, including activities useful for medical, veterinary, and agricultural purposes. The present invention therefore relates to the fields of molecular biology, chemistry, recombinant DNA technology, medicine, animal health, and agriculture.

### BACKGROUND OF THE INVENTION

Polyketides have been produced in a variety of host cells, including Streptomyces, Saccharopolyspora, and Aspergillus for commercial purposes for many years. In particular, these compounds are often found in mycelial bacteria, the actinomycetes, in which the compounds are synthesized by enzymes known as polyketide synthases (PKSs) and produced as secondary metabolites. Typically, a polyketide has been first identified as an active but uncharacterized ingredient in a soil or other environmental sample. Once an active ingredient was identified, the organism that produced the ingredient was isolated and characterized. After the organism was characterized, it was often the subject of an intensive effort to increase the yield of the active ingredient. This effort typically involved successive rounds of subjecting the organism to mutagenic conditions, culturing the mutagenized organisms, and selecting those mutant organisms that produced the active ingredient in higher yields.

Once the genes for the enzymes, called polyketide synthases or PKS(s), that perform the synthesis of polyketides became known, other methods for producing polyketides and improving polyketide production were developed. The PKS enzymes, particularly that class called modular PKS enzymes, that catalyze the synthesis of polyketides are typically very large, multi-subunit proteins encoded by large gene clusters ranging from 10 kilobases (kb) to more than 100 kb in size. See, e.g., PCT patent publication No. 93/13663 (erythromycin); U.S. Pat. No. 6,303,342 B1 (epothilone); U.S. Pat. No. 6,251,636 B1 (oleandolide); PCT patent publication WO 01/27284 A2 (megalomicin); U.S. Pat. No. 5,098,837 (tylosin); U.S. Pat. No. 5,272,474 (avermectin); U.S. Pat. No. 5,744,350 (triol polyketide); and European patent publication No. 791,656 (platenolide), each of which is incorporated herein by reference.

Other advances provided methods by which these and other genes could be transferred to heterologous hosts for the production of the polyketides produced by the products of those genes. See U.S. Pat. No. 5,672,491, incorporated herein by reference. These methods could be used to produce polyketides in any host cell, including cells that naturally produce polyketides but from which the naturally occurring PKS genes had been eliminated and cells that do not naturally produce polyketides. Thus, 6-deoxyerythronolide B (6-dEB, a precursor of erythromycin) was produced in a Streptomyces coelicolor strain from which the endogenous actinorhodin gene cluster had been eliminated. See U.S. Pat. Nos. 5,672,491 and 5,712,146 and McDaniel et al., 1993, Engineered biosynthesis of novel polyketides, Science 262:1546-1550, each of which is incorporated herein by reference. In addition, the successful synthesis of a fungal polyketide, 6-methylsalicylic acid (6-MSA), in E. coli and yeast was reported. See Kealey et al., 1998, Production of a polyketide natural product in nonpolyketide producing prokaryotic and eukaryotic hosts, Proc. Natl. Acad. Sci. USA 95:505-509, U.S. Pat. No. 6,033,883, and PCT patent publication No. 98/27203, each of which is incorporated herein by reference.

Also, methods, reagents, and host cells have been developed for producing polyketides not found in nature. See, for example, U.S. Pat. No. 5,962,290, and PCT publication Nos. 98/49315, both of which are incorporated herein by reference. These methods include methods for producing polyketides using hybrid PKS enzymes and/or synthetic starting units not used by a particular polyketide synthase in nature. See U.S. Pat. Nos. 6,080,555 and 6,066,721 and PCT publication Nos. 97/02358 and 99/03986, each of which is incorporated herein by reference.

A widely used host-vector system, consisting of Streptomyces coelicolor CH999 as host and SCP2*-derived pRM5-based vectors (see U.S. Pat. No. 5,672,491 and McDaniel et al., 1993; a complete citation for each reference cited herein by first author last name and year of publication is located at the end of this section), has proven very useful for the expression of PKS genes. In this system, the cloned genes are expressed under the control of the actI and/or actIII promoters at low copy number and under quasi-natural temporal control, in a host deleted for the actinorhodin (act) gene cluster. The heterologous expression of the entire Saccharopolyspora erythraea 6-deoxyerythronolide B synthase (DEBS) gene cluster (composed of the eryAI, eryAII, and eryAIII genes; see U.S. Pat. No. 5,672,491 and Kao et al., 1994) and the 6-methylsalicylic acid synthase gene of Penicillium patulum (see Bedford et al., 1995) demonstrated that this host-vector system provides an efficient, successful, and expedient platform for heterologous expression and combinatorial biosynthesis.

This host-vector system has also been used to create a large number of “unnatural natural products” (see PCT Pub. No. 98/48315, incorporated herein by reference; McDaniel et al., 1999; Xue et al., 1999; Tang et al., 2000; Tang and McDaniel, 2001), as well as for expressing tailoring gene sets such as those for deoxy-sugar and 3-amino-5-hydroxybenzoic acid biosynthesis (see Wohlert et al., 2001; and Yu et al., 2001). Similar constructs have been expressed in Streptomyces lividans K4-114. While similar in some respects to CH999, the K4-114 strain has the advantage of lacking the methylation-dependent restriction system of S. coelicolor and produces about the same titer of 6-dEB as CH999 when it contains the DEBS gene cluster (see Ziermann and Betlach, 1999; U.S. Pat. No. 6,177,262, incorporated herein by reference; and McNeil et al., 1992).

SCP2*-derived plasmid vectors like pRM5 have a broad host range (see Bibb and Hopwood, 1981; Lydiate et al., 1985, cited below). They have been widely used for heterologous expression, not only in S. coelicolor and S. lividans, but also in Streptomyces parvulus (See Kim et al., 1995), Streptomyces venezuelae, and Saccharopolyspora erythraea (See Rowe et al., 1998).

The parental SCP2* plasmid consists of three functional regions, responsible for replication, partition, and self-transfer (also called the fertility region, because it confers on the plasmid an ability to promote the exchange of chromosomal markers between the parents in a mating). The plasmid also contains a functionally uncharacterized region, between PstI sites 18 and 37 (see FIG. 1 and Kieser et al., 2000, page 265,) absent from all SCP2-based vectors. Both the partition region (see Bibb et al., 1977) and the fertility region (see Lydiate et al., 1985) are important for stable inheritance of SCP2*, the former presumably aiding the distribution of plasmid copies among daughter cells, and the latter presumably ensuring re-infection of any hyphal compartments that fail to inherit plasmid copies.

Most vectors derived from SCP2*, including pRM5, are present in Streptomyces spp. at a low and rather constant copy number of ˜1-5 per chromosome, which makes them very suitable for the cloning of the large gene clusters (>30 kb) typical of multi-module PKS gene clusters. Many high copy number plasmids, such as pU6021 and pU4123, derived from the unrelated p11101, have been developed for the heterologous expression of individual genes in Streptomyces (Takano et al., 1995). However, vectors derived from this plasmid or plasmids containing other high to medium copy number replicons, such as pJV1 or pSG5 (Kieser et al. 2000), have been unsuitable for cloning DNA inserts greater than 30 kb (Xue et al. 1999). Certain SCP2* derivatives, which contain only a part of the replication region (Kieser et al., 2000) have a copy number of around 10, or even many hundreds for pHJL203 (Larson and Hersherger, 1986). Unfortunately, pHJL203 appears to be very unstable. The only stable high copy number SCP2* derivatives so far reported (40-100 copies per chromosome) are co-integrates containing not only the SCP2* replicon but also that of pIJ101. Such co-integrates arose by homologous recombination between SCP2* and pIJ101 derivatives during conjugal transfer but not after co-transformation (Xiao et al., 1994).

Given the valuable nature of many polyketides, there is generally a need to produce polyketides at higher levels and by more efficient processes. This need is compounded by the low titers and yields sometimes observed when PKS genes are transferred to a heterologous host or when a PKS enzyme is altered to produce a novel polyketide. Thus, there is a need for methods, reagents, and host cells that can produce polyketides at higher levels than can be achieved with currently available methodology. There exists a need for stable, high copy number plasmids for heterologous PKS gene expression, a method of producing such plasmids, as well as methods to produce polyketides and other useful products utilizing these plasmids to produce polyketides at much higher levels than can be achieved with currently available methodology. There is also a need for methods to stabilize plasmids for the heterologous expression of PKS genes and the production of polyketides in a host cell. The present invention meets these and other needs.

The following articles provide background information relating to the invention and are incorporated herein by reference. Bedford, D. J., et al., 1995. J. Bacteriol. 177: 4544-4548. Bibb, M. J., et al., 1977. Nature. 284: 526-531. Bibb, M. J., et al., 1981. J. Gen. Microbiol. 126: 427-442. Bibb, M. J., et al., 1994. Mol. Microbiol. 14: 533-545. Blondelte-Rounault, M. H., et al., 1997. Gene. 40: 191-120. Science. 227(5324): 367-369. Henderson, D. J., et al., 1990. Mol. Gen. Genet. 114: 65-71. Kao. C., et al., 1994. Science. 265(5171): 509-512. Kieser, T. J., et al., 2000. Genetic manipulation of Streptomyces: a laboratory manual. John Innes Foundation, Norwich. Khosla, C., et al., 1992. Mol. Microbiol. 6: 3237-3249. Kim, E. S., et al., 1995. J. Bacteriol. 177: 1202-1207. Larson, J. L., et al., 1986. Plasmid. 15: 199-209. Lydiate, D. J., et al., 1985. Gene. 35: 223-235. MacNeil, D. J., et al., 1992. Gene. 111: 61-68. McDaniel, R., et al. 1993. Science. 262: 1546-1550. McDaniel, R., et al., 1999. Proc. Natl. Acad. Sci. USA. 96: 1846-1851. Rowe, C. J., et al. 1998. Gene. 216: 215-223. Schempf, H., et al. 1975. J. Bacteriol. 121: 416-421. Schmitt-John, T., et al., 1992. Appl. Microbiol. Biotechnol. 36: 493-498. Wohlert, S. E., et al., 2001. Chem. Biol. 8(7): 681-700. Tang, L., et al., 2001. Chem. Biol. 8(6): 547-55. Tang L., et al. 2000. Science. 287(5453): 640-642. Takano, E., et al. 1995. Gene. 166: 133-137. van Wezel, W.G. P. et al., 1996. Gene. 182: 229-230. Xiao, J., et al., 1994. Mol. Microbiol. 14: 547-555. Xue, Q., et al., 1999. Proc. Natl. Acad. Sci. USA. 96(21): 11740-11745. Yu, T.W., et al., 2001. J. Biol. Chem. 276(16): 12546-12555. Ziermann, R., et al., 1999. Biotechniques. 26(1): 106-110. Ziermann, R., et al. 2000. J. Ind. Microbiol. Biotech. 24: 46-50.

### SUMMARY OF THE INVENTION

In one embodiment, the present invention provides a plasmid useful for the production of recombinant DNA products in a host cell. In one embodiment, the recombinant DNA product is a DNA, an RNA, a protein, and/or the product of a protein. In one embodiment, the protein is a polyketide synthase (PKS) or a non-ribosomal peptide synthase (NRP S). In one embodiment, the plasmid is useful in the production of a compound produced by a protein, and the compound is a polyketide produced by a PKS that is encoded by a PKS gene cluster on a plasmid. In one embodiment, the compound is a non-ribosomal peptide produced by an NRPS that is encoded by an NRPS gene cluster on the plasmid. In one embodiment, the host cell is an Actinomycetes host cell. In one embodiment, the host cell is a Streptomyces host cell. In one embodiment, the Streptomyces host cell is an S. coelicolor, S. lividans, or S. venezuelae host cell.

In one embodiment, the present invention provides a plasmid useful for the production of a polyketide in a Streptomyces host cell, wherein the plasmid comprises a PKS gene or gene cluster and two replicons derived from SCP2, SCP2*, at least one of which is derived from SCP2@, which differs from SCP2* by a 45 bp deletion described below. In one embodiment, the plasmid also comprises par genes. In one embodiment, the plasmid also comprises tra genes. In one embodiment, the plasmid comprises both par and tra genes. In one embodiment, the plasmid is pSMALL, also known as pKOS97-150.

In one embodiment, the invention provides a method for producing a polyketide, which method comprises: transforming a host cell, said host cell comprising a first plasmid having a first SCP2-derived replicon, with a second plasmid having a second SCP2-derived replicon capable of recombining with the first plasmid to yield a co-integrate plasmid that comprises two SCP2-derived replicons, at least one of which is derived from SCP2@, wherein one of said first or second plasmids further comprises a PKS gene or gene cluster; and culturing said transformed host cell under conditions such that said co-integrate plasmid is formed, said PKS gene or gene cluster is formed, and said polyketide is produced. In one embodiment, the method is practiced with a Streptomyces host cell, such as S. coelicolor or S. lividans, including, but not limited to S. coelicolor CH999 and S. lividans K4-114. In one embodiment, the polyketide is produced by a recombinant PKS. In one embodiment, the recombinant PKS is encoded on the first plasmid in said host cell. In one embodiment, the polyketide is erythromycin or an erythromycin precursor (i.e., 6-deoxyerythronolide B) or an analog or derivative thereof that is produced from a recombinant DEBS or a mutant DEBS that differs from DEBS by substitution, insertion, or deletion of one or more PKS domains and/or amino acid residues. In one preferred mode, the second plasmid is pBoost, also known as pKOS97-188, or pBOOST, also known as pKOS146-145.

In one embodiment, the invention provides a plasmid selected from the group consisting of SCP2@, pKOS97-150 (pSMALL), pBoost, also known as pKOS97-188, or pBOOST, also known as pKOS146-145, and plasmids derived therefrom.

In one embodiment, the invention provides a method of increasing plasmid stability, which method comprises transforming a host cell, said host cell comprising a first plasmid having a first SCP2-derived replicon, with a second plasmid having a second SCP2-derived replicon capable of recombining with the first plasmid to yield a co-integrate plasmid that comprises two SCP2-derived replicons, at least one of which is derived from SCP2@; and culturing said host cell under conditions such that said co-integrate is formed, wherein said co-integrate plasmid is more stable than either of the two plasmids from which it is derived.

In one embodiment, the invention provides a method of producing a polyketide by forming a co-integrate plasmid in a host cell by conjugation, said co-integrate formed from a first vector comprising an SCP2* replicon and a second vector containing a pIJ101 replicon, at least one of said first or second vectors comprising a PKS gene or gene cluster; and culturing said host cell under conditions such that said PKS gene or gene cluster is expressed, and said polyketide is produced. In one embodiment, the invention provides a method of forming a co-integrate plasmid useful in that method by conjugation of two plasmids that share the minimal identical sequence defined by Xiao et al., 1994, supra, wherein at least one of said plasmids comprises a PKS gene or gene cluster.

In one embodiment, the invention provides a plasmid useful for the production of a polyketide, wherein said plasmid comprises a PKS gene or gene cluster and two replicons derived from SCP2, SCP2*, or SCP2@. In one embodiment, the plasmid comprises at least one replicon derived from SCP2@. In one embodiment, the plasmid comprises one replicon derived from SCP2@ and one replicon derived from SCP2*. In one embodiment, both replicons on the plasmid are derived from SCP2@.

These and other embodiments, modes, and aspects of the invention are described in more detail in the following description, examples, and claims set forth below.

### BRIEF DESCRIPTION OF THE DRAWINGS

FIG. 1 is a map of plasmid SCP2@, pBoost (pKOS97-188), and pKOS146-145 (PBOOST) showing the replicons of each.

FIG. 2 illustrates a model for the formation of pKOS97-150 by co-integration between pJRJ2 and the SCP2* variant SCP2@.

FIG. 3 is a schematic of DNA sequence and deduced ORF's of the replicon region of pBoost and pBOOST showing the 45 bp deletion, relative to the replicon region of SCP2*, of each.

### DETAILED DESCRIPTION OF THE INVENTION

In one embodiment, the present invention provides a variant of SCP2*, named SCP2@, which has a 45 bp deletion relative to SCP2*. This deletion is 35 bp upstream of the start codon of ORF1 in the replication region and correlates with enhanced plasmid copy number. The plasmids of the present invention include plasmids that are co-integrates of SCP2@ and SCP2*-derived plasmids. The co-integrates have a high copy number and exhibit increased production of the products of genes contained in them. In one embodiment, these products are PKS and the polyketides produced thereby. Other plasmids of the invention include plasmids pBoost (pKOS97-150) and pBOOST (pKOS146-145), which can be used to form co-integrates with SCP2*-derived vectors and to enhance the production of polyketides and other gene products encoded by the co-integrate plasmids. The present invention provides valuable methods and reagents for the production of useful compounds.

The titer of recombinant 6-deoxyerythronolide B (6-dEB) and 14-desmethyl-6-dEB produced by CH999 carrying plasmids such as pKAO127, which comprises the ery genes that encode DEBS, has been reported to be reproducibly 20-40 mg/L in R5 or R6 medium. Similar titers of 6-dEB analogs such as 15-methyl-6-dEB (also known as 13-propyl-6-dEB; see U.S. Pat. No. 6,274,560) were produced by CH999 carrying pJRJ2 with a KS1 null mutation (Jacobsen et al., 1997) and (2S,3R)-2-methyl-3-hydroxyhexanoate-N-propionyl cysteamine thioester diketide feeding (see U.S. Pat. No. 6,080,555; the KS1 null mutation inactivates the ketosynthase domain of the first extender module and allows the incorporation of supplied precursors, typically diketides, in the polyketide). The titers reported above are similar to those achieved for 6-dEB when the three ery genes (eryAI, eryAII, and eryAIII) were divided between two (Ziermann and Betlach, 2000) or three (Xue et al., 1999) plasmid vectors. Two separate isolates of CH999/pJRJ2 and CH999/pKOS11-26, which were designated KOS120-97 and CH999/pKOS11-26* and used to produce 15-methyl-6-dEB and 6-dEB, respectively, were unexpectedly found to yield 100-160 mg/L of the product in R5 or R6 medium. In a high production medium, KOS120-97 could produce even more 15-methyl-6-dEB (500 mg/L) than its parent strain, CH999/pJRJ2 (20 mg/L).

To examine whether this increased productivity was due to an alteration in the plasmid DNA, total DNA was isolated from KOS120-97 and used to transform E. coli. Two different classes of E. coli XL1-Blue transformants, distinguishable in colony size, were obtained. Plasmids were isolated from both types of transformants. Restriction analysis revealed the same DNA banding patterns and size as pJRJ2 (˜49 kb) for plasmids isolated from the larger colonies (the plasmid actually obtained from these colonies was plasmid pJRJ2Δ, which differs from pJRJ2 by a 45 bp deletion; this was not recognized at that time but only after DNA sequence analysis, as described below), whereas plasmid DNA from most of the smaller colonies had a different banding pattern, indicating a size of ˜80 kb. Further restriction analysis of one of these larger plasmids, pKOS97-150, revealed several bands characteristic of SCP2*-derived vectors and of ery gene sequences, ruling out simple contamination as a source for the novel plasmid.

To see if KOS120-97 contained a mixture of the parental pJRJ2 and the novel plasmid pKOS97-150, genomic DNA was isolated from ten single isolates of KOS 120-97, digested with PstI and BamHI and subjected to Southern blot hybridization with pJRJ2 as probe. All ten isolates showed the same DNA banding pattern as pKOS97-150, different from pJRJ2. This data demonstrated that plasmid pKOS97-150 was the sole plasmid present in KOS120-97 and that pJRJ2 was not present, suggesting that the new plasmid was responsible for the increased polyketide production observed relative to cells having pJRJ2.

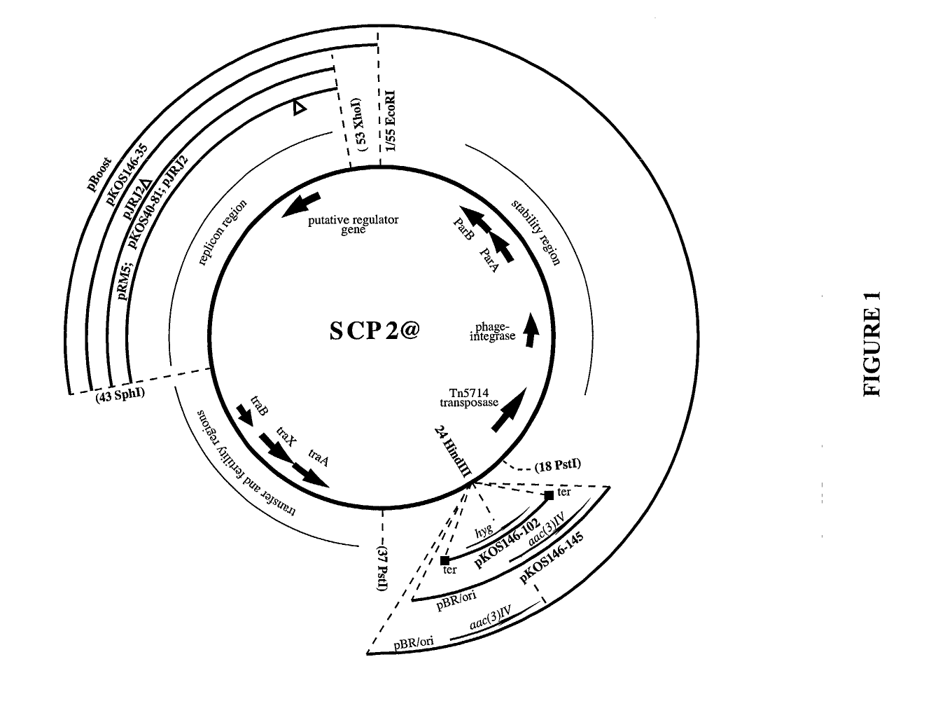

Because S. lividans K4-114 is a suitable alternative host to CH999 for producing recombinant 6-dEB and its analogs, pKOS97-150, pJRJ2, and pJRJ2Δ (FIG. 1) were separately introduced by transformation into K4-114. Strikingly, transformants containing pKOS97-150 produced up to 160 mg/L of propyl-6-dEB, whereas those containing the other two plasmids made only the normal level of 10-20 mg/L. When pKOS97-150 was introduced into the wild-type strain S. coelicolor 1147 by conjugation from CH999/pKOS97-150, the resulting strain had productivity as high as CH999/pKOS97-150. These results demonstrated that the plasmid, and not a genetic change in the CH999 host, was responsible for the high productivity observed.

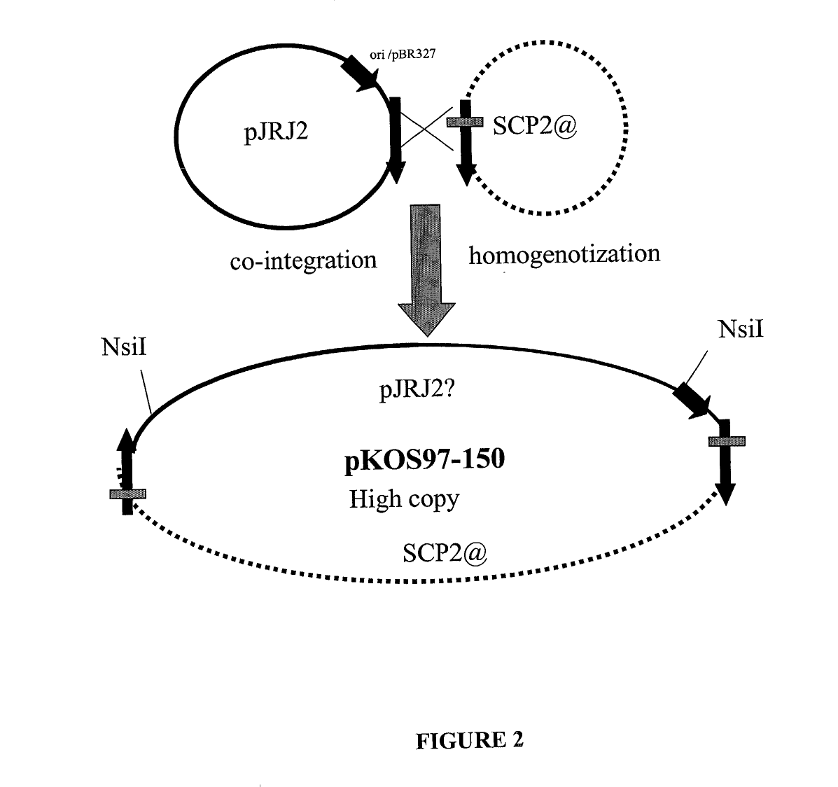

Plasmid pKOS97-174 was constructed to determine the functional elements of pKOS97-150. This plasmid was made by self-ligation of an NsiI fragment of pKOS97-150, taking advantage of the presence of two NsiI sites in pJRJ2, and assuming the absence of NsiI sites from the extra DNA in pKOS97-150 and from the DNA fragment consisting of the actI promoter, actII-ORF4 and the DEBS genes, which are the same as in pJRJ2. Detailed restriction analysis of pKOS97-174 revealed a banding pattern very similar to that of another SCP2*-derived plasmid, pU4231 (Henderson et al., 1990), which all of SCP2* except an ˜7 kb fragment located between the transfer and stability regions (Kieser et al., 2000). Thus pKOS97-150 contains not only the 7.2 kb SCP2* replication region (present in pJRJ2), but also a longer sequence from SCP2*. Random PstI-BamHI fragments from pKOS97-174 were cloned into pUC119 for sequencing. The resulting sequences exactly matched segments of the full SCP2* sequence distributed around SCP2*, including the 7 kb segment not present in pIJ4231 (FIG. 2). The results confirmed that pKOS97-150 contains additional DNA from SCP2* sequences.

Because pKOS97-150 contains sequences of pJRJ2 as well as other segments of SCP2*, and because pJRJ2 and SCP2* share 7.2 kb of identical sequence, pKOS97-150 carries a 7.2 kb duplication (which is believed to explain its resolution in E. coli to pJRJ2Δ, and presumably SCP2*, which cannot replicate in E. coli). Plasmid pKOS97-150 is believed to have been formed by homologous recombination between plasmid pJRJ2 and SCP2*. A physical map of pKOS97-150 was consistent with the digestion patterns generated using BamHI, BglII, EcoRI, EcoRV, PstI and NsiI singly or in combination. FIG. 2 shows a model for the formation of pKOS97-150.

The presence of SCP2* in strain CH999 was unexpected, because SCP2* was believed to have been cured from the parent of CH999. Three isolates of CH999 that had been sub-cultured separately for many transfers, as well as isolates of two of its progenitors, strains B385 and CHi (Khosla et al., 1992), were tested phenotypically for the presence of SCP2-related plasmids. B385, CHI and two isolates of CH999 cultures gave characteristic SCP2 pocks on a lawn of an SCP2⁻ strain, and were resistant to pocking by a control SCP2* strain, while the third CH999 isolate lacked SCP2*. The presence of a plasmid with a size characteristic of SCP2* was confirmed by the isolation of plasmid DNA from a plasmid-containing CH999 isolate.

Plasmids like pJRJ2 normally have a copy number of ˜1-5 per chromosome in S. coelicolor and S. lividans. In contrast, DNA bands of pKOS97-150 were easily visible in agarose gels generated from total DNA of CH999/pKOS97-150 digested with PstI and BamHI, suggesting that pKOS97-150 was present in CH999 at an elevated copy number. An XbaI fragment containing the tsr and glkA genes—which are approximately the same length—from pIJ2581 (van Wezel and Bibb, 1996) was used to measure the relative abundance of tsr on pKOS97-150 and the glkA gene in the host chromosome by Southern blot hybridization. The results confirmed that pKOS97-150 is present in CH999 at ˜100-125 copies per chromosome (estimated by visual comparison), whereas pJRJ2 is present at ˜5-10 copies per chromosome, as expected.

It was previously believed that high copy number is unsuitable for the expression of large PKS gene clusters; however, the results observed with the present invention contradict this belief. Significantly, pJRJ2 was lost very rapidly without selection pressure, whereas pKOS97-150 was relatively stably maintained in its host (0.05% vs. 8.3% of the colonies were thiostrepton resistant after three serial transfers, respectively). It is believed that SCP2* partition and fertility functions both contribute to the apparent stability of pKOS97-150, ensuring that every part of the mycelium remains productive.

These results suggest that when CH999 contains pJRJ2 together with SCP2, SCP2*, or certain other SCP2 derivatives, a co-integrate plasmid capable of high copy number replication can arise by homologous recombination. This hypothesis was tested using three plasmids, each containing a marker gene (for apramycin resistance). These plasmids were pKOS146-105b (which was formed from SCP2*); a pRM5 derived plasmid (pKOS40-81); and pBoost (pKOS97-188, which was derived from pKOS97-150 and contained only one replication region). Plasmid pBoost contains sequences from the SphI site at position 43 to the HindIII site at position 24 of SCP2* (the relevant sites are in FIG. 1; for further details, see Kieser et al., 2000, p. 258). Each plasmid was tested to see if it could increase the productivity of K4-114/pJRJ2 and K4-114/pKOS97-152a. Of the three plasmids, only pBoost could enhance polyketide production and did so in both K4-114/pJRJ2 and K4-114/pKOS97-152a. When R6 medium was used for fermentation, K4-114/pJRJ2, pBoost produced as much 15-methyl-6-dEB as did K4-114/pKOS97-150: an 8-fold increase compared with K4-114/pJRJ2. Similar results were obtained with CH999/pJRJ2 and CH999/pKOS97-152a when transformed with each of the same three plasmids. Table 1 shows increased polyketide production observed with pBoost (pKOS97-188) and a derivative pBOOST, also known as pKOS146-145, constructed as described below.

TABLE 1
Plasmid pBoost - Effect on Polyketide Production

| Strains | Yield of 15-methyl-6dEB | |
|---|---|---|
| K4-114/pJRJ2 | 15-20 | mg/L |
| K4-114/JRJ2/pBoost | 120-160 | mg/L |
| K4-114/pSMALL | 120 | mg/L |
| K4-114/pKOS97-152a | <2.5 | mg/L |
| K4-114/pKSO97-152a/pBoost | 15 | mg/L |
| CH999/pJRJ2 | 15-20 | mg/L |
| CH999/pJRJ2/pBoost | 160 | mg/L |
| CH999/pSMALL | 160 | mg/L |
| CH999/pKOS97-152a | 7 | mg/L |
| CH999/pKOS97-152a/pBoost | 20-35 | mg/L |
| CH999/pKOS97-152a/pBOOST | 40 | mg/L |

After confirmation of the increased titer, plasmids from CH999/pBoost, pJRJ2 and CH999/pBoost, pKOS97-152a were recovered by transforming E. coli XL1-Blue with total DNA from each strain, selecting on apramycin and carbenicillin. Co-integrate plasmids were found in both strains. The copy number of the co-integrate plasmid in the CH999 host was as high as that of pKOS97-150, as determined by Southern blot analysis. It is believed that pBoost (pKOS97-188) carries a mutation in its SCP2 sequences that enables it to form high copy number co-integrates with other SCP2* derivatives, including pJRJ2 and pKOS97-152a; this mutation is a 45 bp deletion, described below.

Plasmid pBoost alone had a high copy number in CH999, as judged by the banding pattern of total DNA cut with PstI and BamH1. When a pBoost-derived plasmid (PKOS146-19) carrying the KS1 null mutation containing DEBS genes under actl promoter control was introduced into K4-114, higher production of 15-methyl-6-dEB (twice the titer conferred by pJRJ2) was observed, but the productivity declined and eventually disappeared after several rounds of propagation. When pKOS146-19 was introduced into CH999 by transformation, most of the transformants were low producers of 15-methyl-6-dEB, but one produced about 100 mg/L. These results indicated that pBoost carrying large inserts was not stable in Streptomyces, even under selection.

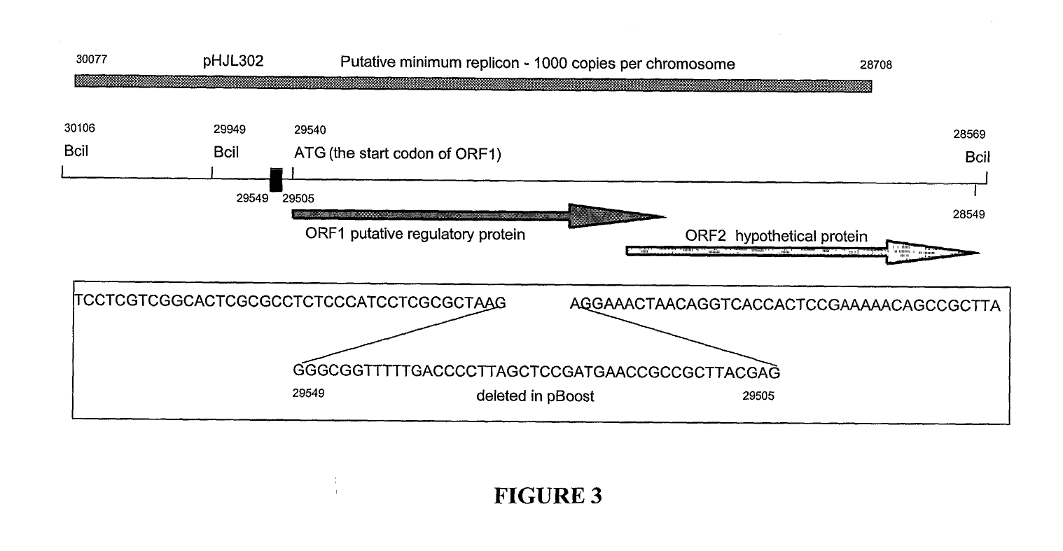

To characterize the special features of pBoost, the plasmid was sequenced. Comparison with the SCP2* sequence revealed a 45 bp deletion in the SCP2 replication region of pBoost (FIG. 3). The deletion was 35 bp upstream of ORFI (Kieser et al., 2000), which is believed to encode a regulatory protein. The deletion may disrupt the promoter for ORF1 transcription. Both replicons on plasmid pSMALL appear to have this deletion. Because the co-integrate plasmid pSMALL was found to carry the 45 bp deletion in both origin regions (replicons), homogenotization may have occurred, either by gene conversion or by reciprocal double crossing over followed by segregation (see FIG. 2 and Kieser et al., 2000).

Plasmid pKOS146-35 was derived from pBoost and lacks the EcoRI-HindIII fragment (sites 1-24) located upstream of ORF1 (FIG. 1). Genes such as those for DEBS (including the KS 1 null variant) under the control of the actI promoter cloned in pKOS146-35 did not result in overproduction, as was observed when cloned in pBoost. Also, the 45 bp deletion was present in pJRJ2-like plasmids (called pJRJ2Δ) isolated from the larger E. coli transformants, and these plasmids likewise did not give rise to high productivity (see below). These results suggest that the EcoRI-HindIII fragment of SCP2 is necessary for increased production. The deletion of this fragment may bring ORF1 fortuitously under the control of a strong promoter.

It was reasoned that the 45 bp deletion must have been present in the SCP2-derived plasmid, called SCP2@, unexpectedly found in CH999 that gave rise to the original co-integrate derivatives that exhibited increased polyketide production. This was confirmed by PCR analysis (data not shown). PCR with different templates, including total DNA of CH999/pKOS11-26* and CH999/pKOS97-150, and plasmid DNA of pKOS97-150, pboost and pJRJ2A, revealed that all the plasmids (pKOS97-150, pKOS11-26*, pBoost, and pJRJ2≢) carried the deletion.

As noted above, further study of plasmid pBoost (pKOS97-188) showed that the increased metabolite productivity of some strains carrying large cloned gene clusters (30 kb in pKOS97-152a) declined upon serial transfer. It was suspected that the unstable productivity might be due to an ˜11 kb fragment present in SCP2@ that carries genes for transfer and fertility but is absent from pBoost (FIG. 1). To test this hypothesis, plasmid pKOS146-102 was made by insertion of a hygromycin resistance gene, Ω hyg (Blondelet-Rouault et al., 1997), into the unique HindIII site (FIG. 1), which is at one end of the SCP2@ sequences in pKOS97-188.

Plasmid pKOS146-102 was introduced along with pKOS97-152a or pJRJ2 by transformation into the CH999 strain, and the resulting transformants, CH999/pKOS146-102, pKOS97-152a and CH999/pKOS146-102, pJRJ2, were fermented as before. Neither of these strains displayed elevated production of 15-methyl-6-dEB compared with the amount produced by the control strains CH999/pKOS97-152a and CH999/pJRJ2.

An attempt was made to create high copy co-integrate plasmids by conjugal mating between CH999/pJRJ2 or CH999/pKOS97-152a and CH999/pKOS146-102 strains. The co-integrate plasmids were isolated in E. coli upon transformation with total DNA from the ex-conjugates, and a slight deviation from the expected restriction pattern was noted. This result suggests that the formation of pKOS97-150 did not result from conjugation as in the case of the high copy plasmid co-integrates described by Xiao et al. (1994). These results also show that, contrary to the results of Xiao et al., two nearly identical SCP2* based plasmid replicons can form high copy number plasmid co-integrates when each plasmid is introduced into the same bacterial host by protoplast mediated transformation or by conjugal transfer and not only by conjugal mating alone as reported by Xiao et al. In one embodiment of the invention, the co-integrate plasmid can be formed by conjugation.

To determine if the apparent lack of high copy number and elevated metabolite productivity might be due to the presence of transcription terminators flanking the hygromycin resistance gene in pKOS146-102, another plasmid was constructed and tested as follows. Plasmid Hu152″ (see Example 1, below) was linearized with HindIII and inserted into the HindIII site of SCP2@ to give pKOS146-145 (FIG. 1). The latter plasmid, which carries the aac(3)IV gene in the same orientation as found in pBoost, performed as well as pBoost: co-integrate plasmids were formed in both the K4-144 and CH999 hosts, and the CH999/pKOS97-152a strains into which pKOS146-145 had been introduced by transformation exhibited an 8 to 12-fold enhancement of 15-methyl-6-dEB production. These results imply that insertion of the hygromycin gene cassette into SCP2@, as in pKOS 146-102, altered the expression of a gene or genes flanking the HindIII site, which somehow affected formation or stability of plasmid co-integrates. In pKOS146-145, in contrast, it is believed that abnormal gene expression did not take place or was affected in a positive manner by read-through from the aac(3)IV promoter.

To confirm whether metabolite production was stable, two representative CH999/pKOS97-152a, pKOS146-145 transformants were grown in liquid TSB medium with only thiostrepton at a concentration that maintains pKOS97-150 in the KOS120-97 strain (see above). After three days, a 100 μl portion of the culture was transferred into fresh growth medium, and the culture was grown for three more days; the rest of the original culture was mixed with an equal volume of 20% glycerol in water and stored at −80° C. This process was repeated twice. The titers of 15-methyl-6-dEB among these serially transferred cultures differed by only a small amount. This result shows that the 11 kb segment of SCP2@ that is missing in pBoost but present in pKOS146-145, named pBOOST, is important for maintaining high metabolite production, most likely by ensuring intercellular plasmid transfer as well as influencing the formation of plasmid co-integrates.

The present invention has important applications to the production of novel polyketides and other compounds in Streptomyces and related host cells. Although it is now becoming routine to generate large numbers of polyketide “unnatural natural products” by genetic engineering, sometimes the levels of production are much lower than those of the original metabolites. Thus technologies for increasing productivity are valuable. The present invention provides a plasmid able to enhance significantly the production of recombinant erythromycin aglycones and other polyketides when allowed to form co-integrates with conventional SCP2*-derived expression plasmids.

Thus, in one aspect, the present invention provides an SCP2* derivative that can be isolated from certain isolates of strain CH999 or otherwise constructed in accordance with the present invention. The CH999 strain was derived from CH1, which was presumed to have been cured of SCP2 (or SCP2*) in its origin from strain B385 (McDaniel et. al., 1993). The curing was performed in a routine fashion (Khosla et al., 1992, and Hopwood et. al., 1985) by introduction of a highly unstable SCP2* derivative, pIJ80, with selection on neomycin-containing agar, followed by spontaneous loss of pIJ80 on a non-selective medium. Evidently, the normal incompatibility between two SCP2-derived replicons must have failed to be expressed in some isolates so that the expected curing did not occur. The 45 bp deletion now known to be present on the ‘surviving’ SCP2 derivative (called SCP2@), which is associated with the special properties described herein, may have allowed it to co-exist with pIJ80, rather than being displaced by that plasmid. However, the fact that an isolate of CH999 could be identified that lacks this or any other SCP2 derivative demonstrates that the endogenous plasmid can be and was cured from S. coelicolor.

The present invention provides SCP2@ (a new SCP2* derivative), plasmids derived therefrom that comprise the novel replicon of SCP2@, and co-integrates between SCP2*-derived plasmids, such as pJRJ2, and SCP2@, which are high-copy number plasmids in the Streptomyces spp. The elevated polyketide production observed when PKS genes are expressed from such high-copy number co-integrates is the result of increased gene dosage and greater plasmid stability. It is believed that the 45 bp DNA deletion upstream of ORF1 in SCP2@ is responsible for the increased copy-number of the co-integrate plasmids, and that gene(s) flanking the HindIII site govern co-integration between SCP2@ (and its derivatives) and SCP2*-derived plasmids. It also is likely that genes in the region of plasmid transfer and fertility (FIG. 1) are important to maintain high metabolite productivity upon expression of large PKS gene clusters in heterologous hosts. Plasmids pBoost (pKOS97-188) and pBOOST (pKOS146-145) can mimic SCP2@ and form co-integrates with SCP2*-derivatives. Both plasmids can be used to achieve a large increase in metabolite production from cloned PKS and other types of genes, as a consequence of the co-integrate plasmids formed during protoplast-mediated transformation. A detailed description of the invention having been provided above, the present invention will be illustrated as embodied by the following non-limiting examples.

### EXAMPLE 1

### Plasmids, Strains, and Culture Conditions

Streptomyces coelicolor CH999, described in WO 95/08548, or S. lividans K4-114 or K4-155 described in Ziermann and Betlach, 1999, each of which is incorporated herein by reference, were used as expression hosts. Escherichia coli XL-1 Blue, available from Strategene, was used as the E. coli host. Many of the expression vectors of the invention illustrated in the examples are derived from plasmid pRM5, described in WO 95/08548. This plasmid includes a colE1 replicon, an appropriately truncated SCP2* Streptomyces replicon, two act promoters, the actI and actIII promoters, to allow for bi-directional cloning, the gene encoding the act-II-ORF4 activator, which induces transcription from act promoters during the transition from growth phase to stationary phase, and appropriate marker genes. Engineered restriction sites in the plasmid facilitate the combinatorial construction of PKS. When plasmid pRM5 is used for expression of a PKS, all relevant biosynthetic genes can be plasmid borne and therefore amenable to facile manipulation and mutagenesis in E. coli. This plasmid and its derivatives are also suitable for use in Streptomyces host cells. Streptomyces is genetically and physiologically well characterized and expresses the ancillary activities required for in vivo production of most polyketides. Plasmid pRM5, SCP2* and derivatives utilize the act promoter for PKS gene expression, so polyketides are produced in a secondary metabolite-like manner, thereby alleviating the toxic effects of synthesizing potentially bioactive compounds in vivo.

The Streptomyces and E. coli strains and plasmids used in these examples are shown in Table 2 below.

TABLE 2
Bacterial Strains and Plasmids

| Strains/plasmids | Genotype/relevant characteristics | Reference/source |
|---|---|---|
| Strains | | |
| E. coli X11-Blue | F′::Tn10,proA⁺B⁺,lacl^{q}Δ(lacZ)M15/recA1, endA1, | Stratagene, La Jolla, |
| | hsdR17(rₖ⁻mₖ⁻), glnV44, relA1 | CA, USA |
| E. coli ET12567 | dam, dcm, hsdS, cam^{r}, tet^{r} | MacNeil et al. (1992) |
| S. coelicolor CH999 | proA1, argA1 redE60 Δact::ermE SCP⁻, SCP2⁻ | McDaniel et al. |
| | | (1993) |
| S. lividans K4-114 | str-6, SLP2⁻, SLP3⁻, Δact::ermE | Ziermann and Betlach |
| | | (1999) |
| CH999/pKOS11- | 6-dEB high-production isolate of | This work |
| 26* | CH999/pKOS11-26 | |
| KOS120-97 or | 15-methyl-6-dEB high-production isolate of | This work |
| CH999/pKOS97- | CH999/pJRJ2 | |
| 150 | | |
| CH999/SCP2@ | proA1, argA1 redE60 Δact::ermE SCP⁻, SCP2@ | This work |
| Plasmids | | |
| pKOS97-150 | Co-integrate between SCP2@ and pJRJ2Δ | This work |
| pJRJ2 | pRM5 derivative carrying DEBS with KS1 null | Jacobsen et al. (1997) |
| | mutant | |
| KAO127 | pCK7 with kanamycin resistance gene inserted in | Kao et al. (1994) |
| | HindIII, produced as much 6-dEB as pCK7. | |
| pKOS40-81 | pRM5 with apramycin (aacIV3) resistance gene | This work |
| | inserted into tsr gene | |
| pJRJ2Δ | As pJRJ2, but with 45 bp deletion in SCP2* | This work |
| | replication region, isolated from the big E. coli | |
| | transformants. | |
| pKOS97-174 | NsiI fragment of pKOS97-150 produced by self | This work |
| | ligation | |
| pBoost (pKOS97- | Hu152′ with the HindIII-EcoRV fragment | This work |
| 188) | replaced by the corresponding fragment from | |
| | pKOS97-174 | |
| Hu152′ and Hu152″ | pBR322 derivative with bla gene replaced by | This work |
| | aac(3)IV; in Hu152″ the NdeI site was filled in by | |
| | treatment with T4 DNA polymerase | |
| SCP2* | High-fertility variant of SCP2, tra⁺, par⁺ | Bibb et al. (1977) |
| SCP2@ | SCP2* derivative, containing 45 bp deletion in | This work |
| | replication region | |
| pKOS97-152a | pRM5 derivative, carrying modified meg PKS | This work |
| | gene with KS1 null mutation | |
| pKOS146-105b | Hu152′ inserted into EcoRI site of SCP2* | Kosan Biosciences |
| pKOS146-35 | Self-ligation product of EcoRI-digested pBoost | Kosan Biosciences |
| pKOS146-19 | pBoost derivative, carrying DEBS genes with | Kosan Biosciences |
| | KS1 null mutant under actI promoter control | |
| pKOS146-102 | A HindIII fragment carrying Ωhyg inserted into | Kosan Biosciences |
| | HindIII site of SCP2@ | |
| pKOS146-145 | Hind III linearized Hu152″ inserted into HindIII | Kosan Biosciences |
| (pBOOST) | site of SCP2@ in the orientation shown in Figure 1 | |

For Streptomyces strains, R5 medium was used for propagation and protoplast transformation. TSB medium was used to culture Streptomyces strains for DNA preparation and as seed medium; R6 medium was used for fermentations. For E. coli, LB medium was used for all purposes. The recipes for R5, TSB, LB and trace element solution in R6 medium are from Kieser et al. (2000). R6 medium consists of 103 g/L sucrose, 0.25 g/L K₂SO₄, 10.12 g/L MgCl₂.6H₂O, 0.96 g/L sodium propionate, 0.1 g/L Difco casamino acids, 5.0 g/L yeast extract, 28.2 g/L Bis-Tris propane and 2.0 ml trace element solution. After autoclaving, the following were added per liter: 10 ml KH₂PO₄ (0.5% w/v), 8 ml CaCl₂.2H₂O (2.5 M) and 15 ml L-proline (20%, w/v). The final concentration of antibiotics used in all media was 60 mg/L of apramycin, 50 mg/L of carbenicillin, 25 mg/L of chloramphenicol, 50 mg/L of kanamycin, and 50 mg/L of thiostrepton. The diketide was (2S,3R)-2-methyl-3-hydroxyhexanoate N-propionyl cysteamine thioester (“propyl diketide”).

Polymerase chain reaction (PCR) was performed using Pfu polymerase (Strategene; Taq polymease from Perkin Elmer Cetus can also be used) under conditions recommended by the enzyme manufacturer. Standard in vitro techniques were used for DNA manipulations (Sambrook et al. Molecular Cloning: A Laboratory Manual (Current Edition)). E. coli was transformed using standard calcium choloride-based methods; a Bio-Rad E. coli pulsing apparatus and protocols provided by Bio-Rad could also be used. S. coelicolor was transformed by standard procedures (PEG-mediated, protoplast transformation; Hopwood et al., 1985).

Primers 146-92F5 and 146-92B11-F5 were used to detect the 45 bp deletion in the SCP2 replication region. Their sequences are:

| | |
|---|---|
| 146-92F5 | [5′-d(GGCTCCCTCCCAGATTCG)-3′; and |
| 146-92B11-F5 | [5′-d(GGTGACCTGTTAGTTTCCTCTCG)-3′. |

These primers were adapted from Hopwood et al. (1985) and Kieser et al. (2000).

Streptomyces transformants were picked into 6 ml of TSB liquid medium with 25-50 mg/L of the appropriate antibiotic and grown at 30° C. After sufficient growth (normally 3-4 days), they were transferred to 250 ml flasks containing 50 ml of R6 medium (supplemented with the appropriate antibiotics and the propyl diketide at a final concentration of 1 g/L). The flasks were shaken at 30° C. for about 7 days, after which 1 ml of culture was withdrawn and spun down. A 200 μl sample of the supernatant, or a dilution of it, was subjected to HPLC/MS analysis. Analysis and quantitative determination were according to Xue et al. (1999), with unpublished modifications, using 15-methyl-6-dEB as standard. See U.S. Pat. No. 6,274,560 (15-methyl-6-dEB), U.S. Pat. No. 6,080,555 (pJRJ2), and U.S. Pat. Nos. 6,066,721, 6,261,816, and PCT Publ. 99/03986 (diketide feeding) incorporated herein by reference.

### EXAMPLE 2

### Increased Polyketide Production with Plasmids pBoost and pBOOST

Plasmid pBoost enhances polyketide production in both K4-114/pJRJ2 and K4-114/pKOS97-152a. When R6 medium was used for fermentation, K4-114/pJRJ2, pBoost produced as much 15-methyl-6-dEB as did K4-114/pKOS97-150 (pSMALL): an 8-fold increase compared with K4-114/pJRJ2. Similar results were obtained with CH999/pJRJ2 and CH999/pKOS97-152a when transformed with each of pBoost and pBOOST. Table 3 shows increased polyketide production observed with pBoost (pKOS97-188) and further pBOOST (pKOS146-145) in CH999 cells.

TABLE 3
Effect of pSMALL, pBoost, and pBOOST on Polyketide Production

| Strains/Plasmids | Yields of 15-methyl-6dEB | |
|---|---|---|
| K4-114/JRJ2/pBoost | 120-160 | mg/L |
| K4-114/pSMALL | 120 | mg/L |
| K4-114/pKSO97-152a/pBoost | 15 | mg/L |
| CH999/pJRJ2/pBoost | 160 | mg/L |
| CH999/pKOS97-152a/pBoost | 20-35 | mg/L |
| CH999/pKOS97-152a/pBOOST | 40 | mg/L |

The foregoing description and non-limiting examples of the present invention describe SCP2@ (a new SCP2* derivative) and a co-integrate between pJRJ2 and SCP2@, named pKOS97-150 (pSMALL), which are high-copy number plasmids in the Streptomyces spp. In addition to SCP2@ and pSMALL, the present invention provides pBOOST, also known as pKOS146-145, and pBoost, also known as pKOS97-188, the former plasmid having an additional segment that mediates self transfer of the plasmid. These two plasmids can be used directly as expression vectors for PKS and other genes that are inserted into them or, in a preferred embodiment, to form co-integrate plasmids with other SCP2-derived vectors that exhibit elevated gene expression, increased copy number, and greater plasmid stability.

SCP2@ differs from the known and publicly available plasmid SCP2* in that it has, relative to the latter plasmid, a 45 bp deletion upstream of ORF 1. This deletion contributes to the increased copy-number of the co-integrate plasmids, and gene(s) flanking the HindIII site govern co-integration between SCP2@ (and its derivatives) and SCP2*-derived plasmids. Genes in the region of plasmid transfer and fertility (FIG. 1) are also important to maintain high metabolite productivity upon expression of large PKS genes in heterologous hosts. Plasmids pBoost (pKOS97-188) and pBOOST (pKOS146-145) can mimic SCP2@ and form co-integrates with SCP2*-derivatives. Both plasmids can be used to achieve a large increase in metabolite production from cloned PKS and other types of genes as a consequence of the co-integrate plasmids formed during protoplast-mediated transformation.

Thus, the present invention provides a method of increasing production of plasmid-borne genes in Streptomyces and related host cells (cells in which Streptomyces plasmids replicate and in which Streptomyces promoters function) by forming co-integrate plasmids that comprise at least two SCP2*-derived replicons, at least one of said replicons having the 45 bp deletion by which SCP2@ differs from SCP2*, in said host cells, and culturing said host cells comprising said co-integrate plasmids under conditions such that said plasmid-borne genes are expressed.

Although the present invention has been described in substantial detail with reference to specific embodiments, those of skill in the art will recognize that changes may be made to the embodiments specifically disclosed within the scope and spirit of the invention, as set forth in the claims that follow. Numerous modifications may be made to the foregoing teachings without departing from the invention. All publications or patent documents cited in this specification are incorporated herein by reference as if each such publication or document was specifically and individually indicated to be incorporated herein by reference. Citation of the above publications or documents is not intended as an admission that any of the foregoing is pertinent prior art, nor does it constitute any admission as to the contents or date of these publications or documents.

What is claimed is:

1. A plasmid comprising at least two replicons, one of which is derived from SCP2 or SCP2* and one of which is derived from SCP2@.

2. The plasmid of claim 1 that also comprises one or more genes selected from par and tra genes.

3. The plasmid of claim 1 that also comprises one or more polyketide synthase genes or non-ribosomal peptide synthase genes.

4. The plasmid of claim 1 that is pSMALL (pKOS97-150).

5. A plasmid comprising a replicon of SCP2@.

6. The plasmid of claim 5 that is selected from the group of plasmids consisting of SCP2@, pBoost (pKOS97-188), and pBOOST (pKOS146-145).

7. A method for producing a polyketide, which method comprises: transforming a host cell, said host cell comprising a first plasmid having a first SCP2-derived replicon, with a second plasmid having a second SCP2-derived replicon and capable of recombining with the first plasmid to yield a co-integrate plasmid that comprises at least two SCP2-derived replicons, wherein either said first or second plasmid comprises one or more polyketide synthase genes and wherein one of said SCP2-derived replicons is derived from SCP2@; and culturing said host cell under conditions such that said recombination occurs and said PKS genes are expressed so as to produce a functional PKS that produces said polyketide.

8. The method of claim 6, wherein said host cell is a Streptomyces host cell.

9. The method of claim 6, wherein said second plasmid is pBoost (pKOS97-188) or pBOOST (pKOS146-145).

10. A method of forming a co-integrate plasmid having increasing plasmid stability relative to plasmids from which said co-integrate is derived, which method comprises transforming a host cell, said host cell comprising a first plasmid having a first SCP2-derived replicon, with a second plasmid having a second SCP2-derived replicon capable of recombining with the first plasmid to yield a co-integrate plasmid that comprises at least two SCP2-derived replicons, wherein said first and second plasmids comprise one or more polyketide synthase genes and wherein one of said SCP2-derived replicons is derived from SCP2@; and culturing said host cell under conditions such that said recombination occurs.

11. The method of claim 10 wherein one of said first and second plasmids is selected from the group of plasmids consisting of pBoost (pKOS97-188) and pBOOST (pKOS146-145).
